(19) United States
(12) Patent Application Publication
(10) Pub. No.: US 2007/0280997 A1
(43) Pub. Date: Dec. 6, 2007

(54) Composition for enhancing muscle recovery

(75) Inventor: Robert Portman, Fair Haven, NJ (US)

Correspondence Address: GIBBONS P.C., ONE GATEWAY CENTER, NEWARK, NJ, 07102, US

(73) Assignee: PacificHealth Laboratories, Inc.

(21) Appl. No.: 11/444,177

(22) Filed: May 31, 2006

(51) Int. Cl. A61K 31/70

(52) U.S. Cl. 424/439; 514/23; 514/53

(57) ABSTRACT

An essential composition that acts to prevent or retard loss of muscle strength and atrophy during extended periods of inactivity is provided. The compositions are characterized by a ratio of carbohydrate to protein of from about 1.5:1 to 2:1 and by the presence of a specific amino acid component of leucine and, optimally, argilline. The carbohydrate component is at least two members of a group of four sugars and the protein component is whey protein and casein. A preferred carbohydrate component is glucose and sucrose. The ratio of whey protein to casein in the subject compositions is about 3:1. The subject compositions are preferably provided in preparations containing conventional excipient materials such as flavors, coloring agents, emulsifiers, artificial sweeteners and the like, such preparations providing from about 33 to 58 grams of the essential composition, which comprises from about 15% to 80% by weight of said preparations.

### FIELD OF INVENTION

The present invention relates to a nutritional composition for enhancing muscle recovery following surgery or during extended periods of inactivity.

### BACKGROUND OF THE INVENTION

Following exercise it has been shown that consumption of a carbohydrate and protein supplement can stimulate a number of anabolic activities which can result in enhanced muscle recovery and a reduction in muscle damage. The reason for this enhanced anabolic activity is that, following exercise, the muscle cells are highly responsive to the hormone insulin. The stimulation of insulin initiates or enhances a number of anabolic activities in the body, including transport of glucose into muscle cells, transport of amino acids into muscle cells, synthesis of muscle glycogen, synthesis of protein and increased blood flow to the muscles. When insulin is stimulated post-exercise in the presence of carbohydrate and protein, there is a significant increase in muscle recovery, a decrease in muscle damage and a greater level of endurance in a subsequent exercise bout. Typically, the insulin responsiveness of muscles peaks about thirty to forty minutes following the cessation of exercise.

In addition to stimulating insulin, carbohydrate-protein supplements also increase a number of protein synthetic pathways, which result in greater muscle growth. In my previous patent, No. 6,051,236, it was shown that ingestion of a composition containing a combination of carbohydrate and protein in a weight ratio of from about 2.8 to 4.2 to 1 produces a stimulation of muscle anabolic activity via insulin with substantially no interference with gastric emptying. The balance between anabolic stimulation and no interference with gastric emptying is particularly important following exercise since hydration is an essential component of muscle recovery. U.S. Pat. No. 6,051,236 teaches the importance of including electrolytes, such as sodium, potassium, magnesium and chloride, in a recovery beverage to replenish those lost during sweating. When gastric emptying is slowed, however, the recovery processes related to rehydration are also slowed, leading to an overall decrease in muscle recovery. Another important consideration in the use of a supplement following exercise is the need to replenish muscle glycogen stores. The ratio of carbohydrate to protein stated above is also advantageous following exercise as the carbohydrate not only serves as an anabolic stimulus for protein synthesis, but is essential to replenish muscle glycogen stores.

While the emphasis in the formulation of supplements such as described above has been on their use during and following exercise to aid the body in rehydration and muscle recovery, there is clearly a need in the art for a nutritional formulation specifically designed for use by those individuals whose muscles lose strength and atrophy. These manifestations may result from a variety of surgical procedures, illness or forced inactivity, for example to recover from injuries such as sprains that do not require surgery, yet force the individual to be inactive for an extended period of time. Such loss of strength and muscle atrophy typically necessitates extensive periods of rehabilitation involving physical therapy to aid the affected muscles regain strength, function and tone. These symptoms are often seen in the elderly who must undergo extended periods of inactivity as a result of a variety of afflictions and illnesses as well as injuries. In the elderly, these symptoms can have major economic implications as the time and cost required for recovery and therapy are markedly increased if the involved muscles have had the opportunity to significantly weaken and atrophy before therapy can be begun.

An additional complication of inactivity following surgery, illness or inactivity for extended periods of time is the potential for the individual to develop deep vein thrombosis. It is recognized that certain individuals, again the elderly being prominent among them, are more prone to the development of thrombosis than others. Insulin stimulation has been shown to be beneficial in this regard in that it produces an increase in blood flow to muscles and may reduce the potential for deep vein thrombosis to occur.

Previous research conducted on individuals who underwent forced bed rest have shown that a nutritional composition containing essential amino acids(EAA) and sticrose(carbohydrate) in a ratio of approximately 1:2 (EAA-carbohydrate) was effective in increasing muscle strength and slowing the rate of muscle atrophy. The formulation used in this study suffers from a number of deficiencies. Although essential amino acids are effective in initiating muscle protein synthesis, the presence of only essential amino acids, in the formulation may actually decrease the overall amount of new protein synthesized since non-essential amino acids are also critical in the process. A formulation that contains whole protein that has a good essential amino acid profile is not only more effective but also would be far more economical, an important consideration in the development of any health care product. Furthermore, the formulation contained only sucrose as the carbohydrate component. The use of sucrose has been shown to be effective in stimulating insulin, a hormone critical for promoting protein synthesis. However, the use of a combination of carbohydrates is far more effective in stimulating insulin release since there are multiple and distinct carbohydrate transporters in the gastrointestinal tract that would be involved if additional sugars were present. The use of a carbohydrate combination rather than a sole sugar, such as sucrose, results in a more efficient transport into the blood and a greater stimulation of insulin which translates into greater protein synthesis.

U.S. Pat. No. 4,780,475 teaches the use of a nutritional composition to prevent muscle catabolism and increase protein synthesis in individuals undergoing severe metabolic stress. This patent discloses a formulation of purified essential and non-essential amino acids to help reduce muscle catabolism. Although it is stated that the amino acid compositions may be taken with other nutrients including carbohydrates, the only example given show a carbohydrate to protein ratio of greater than 5:1. At such a ratio, individuals would be consuming an excessive amount of carbohydrate calories. Because they are inactive, there would be additional conversion of the excess carbohydrate into fat. Furthermore, the use of purified amino acids rather than whole proteins will increase the cost of such a composition.

U.S. Pat. No. 5,276,018 discloses a combination of essential and non-essential amino acids, having a specific content of branched-chain amino acids, to help prevent metabolic tissue breakdown. The disclosed formulations may include from about 10 to 25% of glucose. It is noted that glucose is reported in the literature to inhibit some levels of catabolic tissue breakdown and that its presence decreases the release of alanine from skeletal muscle. There is nothing that in any way would teach or suggest the criticality of a particular range of carbohydrate to protein. AS was the case with the patent discussed above, the use of purified amino acids significantly increases the cost of the preparation.

Accordingly, there is clearly a need for a nutritional composition that can maximize protein synthesis following surgery, illness or extended periods of forced inactivity resulting in loss of muscular strength and atrophy. It is further desirable that such a composition will act to increase peripheral blood flow to muscles, thereby potentially minimizing the risk of development of deep vein thrombosis. It is also highly desirable that such a composition be low in calories and economical in cost. Such a composition is provided in accordance with the present invention.

### SUMMARY OF THE INVENTION

In accordance with the present invention, there is provided a specific composition that acts to retard loss of muscle strength and atrophy during an extended period of inactivity. The essential compositions of the invention are characterized by a weight ratio of carbohydrate to protein of from about 1.5:1 to 2:1 and by the presence of a specific amino acid component of leucine and, optimally, arginine. The carbohydrate component is a mixture of at least two sugars selected from the group of glucose, fructose, sucrose and maltodextrin. The protein component is a mixture of whey protein and casein in a weight ratio of about 3:1. Preparations containing the essential compositions of the invention may also contain conventional excipient ingredients, such as flavoring agents, emulsifying agents and the like. Such preparations may be provided in liquid form ready for consumption, powder form to be mixed with a suitable liquid or added to a food before consumption, or a nutrient bar or similar preparation.

### DETAILED DESCRIPTION OF THE INVENTION

In accordance with the present invention, it has been found that selective carbohydrates and protein in a specific weight ratio of from about 1.5:1 to 2:1, combined with a specific amino acid component, form a unique essential composition that is useful for retarding muscle atrophy and loss of strength resulting from extended periods of inactivity, such as would be necessitated by surgery, injury or illness. The subject essential compositions are unique in that they maximize and extend the synthesis of protein thereby retarding the loss of muscle strength and tone and preventing atrophy, even in the absence of stimulation by exercise. The subject compositions are advantageous in that, unlike conventional preparations intended to aid muscle recovery from exercise, they are specifically formulated to aid and enhance muscle recovery where the patient is incapable of exercising the affected muscles.

While the subject compositions are in some ways similar to the compositions disclosed in U.S. Pat. No. 6,051,236, as well as in certain compositions disclosed in the literature as discussed above, they differ in critical ways that make them uniquely advantageous for use by individuals who suffer from, or have the potential to undergo, muscle weakness and atrophy as the result of extended inactivity due to surgery, injury, illness or similar causes. It has unexpectedly been found that a specific weight ratio of from about 1.5:1 to 2:1 carbohydrate to protein, in combination with a specific amino acid component described below, renders the subject essential compositions particularly advantageous for treating or preventing muscle weakness and/or atrophy resulting from extended inactivity.

A major difference between the biochemical state of muscles post-exercise versus that resulting from a period of forced inactivity is related to the insulin responsiveness of the muscle cells. Muscle cells post-exercise have a heightened responsiveness to insulin, which is not found in muscle cells following a period of forced inactivity. As a result of this essential finding, the compositions of the present invention are formulated to include specific nutrients that stimulate and maintain insulin levels to enhance muscle anabolic activity.

The carbohydrate component of the essential compositions of the present invention consists of at least two sugars selected from the group consisting of sucrose, glucose, fructose and maltodextrin. The carbohydrate component improves the palatability of the subject compositions. This is advantageous in terms of patient compliance since, typically, it may be necessary for an individual to take the subject compositions for a number of weeks, for example, where a member is immobilized in a cast, or where major surgery forces inactivity. Optimally, the two or more sugars making up the carbohydrate component are present in approximately equal quantities although they may be present in a weight ratio of up to about 10 to 1. Where more than two sugars are present, it is preferred that they be in equal quantities. A preferred carbohydrate component is a mixture of approximately equal quantities of glucose and sucrose. The essential composition according to the present invention, on a dry weight basis, contains from about 35% to about 70%, preferably from about 45% to about 66% by weight of the carbohydrate component.

The protein component of the essential compositions of the present invention comprises a combination of whey and a casein proteins. The whey protein can be in any of the commercially available forms, such as the concentrate, hydrolysate or isolate. Likewise, casein may be in commercially available form, such as calcium caseinate. The relative proportion of whey protein to casein should preferably be a weight ratio of about 3:1 and should not exceed about 9:1. This combination of proteins is optimum in providing an extended level of protein synthetic activity in that the whey protein produces a fast-acting effect, whereas the caseinate produces a more sustained effect. While whey protein is preferred in the subject compositions, other fast-acting proteins, such as soy protein, may be used, for example, where the patient has demonstrated an allergic reaction to milk proteins. The total protein concentration in the essential composition of the present invention is from about 25% to 35%, preferably from about 30% to 33% by weight, based on the dry composition.

The specific carbohydrate to protein ratio of about 1.5:1 to 2:1 in the essential compositions of the subject invention is necessary to increase blood amino acid levels, thereby stimulating insulin production to maintain a high level of glucose and amino acids into the muscle cells and stimulate muscle glycogen synthesis. Increased blood amino acid levels independently increase protein synthesis by activating protein translation, an essential step in the manufacture of protein. These anabolic activities strengthen the muscles against weakening and atrophy that would occur were the level of these activities to drop as a result of inactivity over extended periods of time. In contrast, a preparation to be taken following exercise will ideally contain a higher ratio of carbohydrate to protein because the cells are in an insulin responsive state.

An essential difference in the use of the subject compositions to enhance muscle recovery and prevent or minimize muscle atrophy following inactivity for an extended period of time versus a preparation used after exercise is the lower carbohydrate to protein content of the subject compositions. Consuming higher levels of carbohydrate during a period of forced inactivity could result in weight gain since the individual in the large majority of instances will not be able to partake in the physical activity and exercise necessary to burn off the carbohydrate calories.

The subject essential compositions additionally contain an amino acid component consisting of leucine and, optimally, arginine. The branched-chain amino acid leucine compliments the protein-carbohydrate components because it is a potent activator of muscle growth. It serves not only as a building block for muscle proteins, but helps amplify muscle protein synthesis via three mechanisms. First, leucine increases blood insulin levels by stimulating the release of insulin from the pancreas. Second, it works in cooperation with insulin to initiate protein synthesis, and third, leucine increases protein synthesis by stimulating translation mechanisms. Leucine is present in the compositions of the invention in from about 6% to 9% by weight, preferably from about 7% to 8% by weight, based on the dry weight of the essential composition

The addition of arginine provides two important benefits to the subject compositions. Arginine is an effective stimulator of insulin release. The inclusion of arginine provides a second mechanism to create the necessary insulin levels that drive protein synthesis and thus enables the carbohydrate content to be further reduced thereby resulting in a calorically efficient preparation. Individuals who undergo periods of forced inactivity following orthopedic surgery or injury expend less calories per day. Reducing the total amount of calories in the subject preparations also decreases the potential conversion of unused calories into unwanted weight gain. Furthermore, a natural consequence of forced inactivity is a reduced blood flow to muscles, particularly where a limb is immobilized, as in a cast. When blood flow is reduced, there is a decrease in the amount of nutrients received by muscle cells and, therefore, a decrease in muscle synthetic activity. Arginine increases the flow of blood to muscle cells by stimulating nitric oxide mechanisms. This is an important benefit to individuals who are undergoing forced inactivity following injury, illness or surgery. Arginine is optimally present in the essential compositions of the invention in from about 3% to 9% by weight, preferably about 7% to 8% by weight, based on the dry composition.

Preparations containing the essential compositions on the subject invention for consumption may be in the form of a dry powder that can be utilized by an individual in need thereof in a number of ways, or in a prepared liquid drink or solid form, such as an nutrient bar. In either instance, such preparations will contain conventional flavoring and sweetening agents and suitable coloring agents to compliment the flavor imparted thereto, for example a yellow color would be utilized with a banana flavor and the like. The preparations may also contain one or more of emulsifiers, fillers, binders, wetting agents and suitable liquid or solid vehicles for the administration of foods. For incorporation into a liquid preparation to form a drink, there may be included emulsifiers, including but not limited to, lecithin and phosphatidyl choline derivatives, acacia, or veegum and one or more surfactants, particularly non-ionic surface active agents, for example the Tween series.

The preparations containing the subject essential compositions may likewise include coloring agents, or pigments, such as FD&C or D&C approved lakes and dyes, iron oxide and titanium dioxide and artificial sweeteners, such as aspartame, sodium cyclamate and sodium saccharinate. The flavoring component may include, without intended limitation, water soluble, natural or artificial extracts of apple, banana, cherry, cinnamon, cranberry, grape, honeydew, kiwi, lemon, lime, orange, peach, peppermint, pineapple, raspberry tangerine, watermelon, wild cherry and the like.

Preparations containing the subject essential compositions in powder form are preferably formulated to be combined with a food or foods, or more preferably, a suitable liquid such as water or a juice. It is also contemplated that such preparations may be formulated pre-mixed with a liquid or solid vehicle suitable for the administration of foods, such as a prepared drink, or in a bar, such as commercial power bars. Regardless, it is contemplated such preparations will comprise from about 15% to about 80% by weight of the essential composition as described above with the remainder being conventional excipient materials recognized as being useful in such preparations, also as described above. The amount and selection of the type of excipients would depend on the desired final form of the preparation and are considered to be within the purview of a person skilled in the relevant compounding or formulating arts.

The essential compositions of the present invention are preferably administered in a plurality of doses over the course of a day to achieve maximum benefit, for example, with meals. It is considered to be of greater benefit in the prevention of loss of muscle strength and atrophy that administration of the subject compositions be at intervals over a day rather than a larger dosage given in a single administration. The subject composition are equally important for their preventative capacity where there is the potential for muscle atrophy as for their capacity to neutralize or reverse to some extent the process of loss of muscle strength or atrophy. Therefore, the compositions of the present invention are indicated where an individual, particularly an elderly individual, has undergone surgery, or sustained an injury, or is afflicted With an illness that would dictate an extended period of inactivity, even in those situations like an injury to a limb, where only a portion of the body is immobilized.

Although the dosage regimen may be varied depending on the needs of the individual, the amount of the compositions of the present invention to be administered to a patient in need thereof in a single dosage is typically sufficient to provide from about 33 to 58 grams of the essential composition For most individuals in need of the essential compositions of the present invention, single dosages may be taken three times daily. While it is possible to take additional amounts on a daily basis, or take the total amount in a single daily dosage, three dosages over the course of a day, for example with meals, is recommended. Individual dosages in prepared form, whether in liquid or solid form, should contain from about 39 to 47 grams of the essential composition. Likewise, directions for use of a prepared powder to be added to food or, preferably, a liquid such as water or juice, should instruct the user to add a sufficient of the powder to provide the recommended dosage.

Throughout this application, there has been reference to an extended period of inactivity. How long it takes for muscles to begin to lose strength and subsequently begin to atrophy varies widely according to a number of factors including the age and general health and metabolism of the individual, the muscle strength and muscle tone of the individual, the surgical procedure, illness or injury requiring the forced inactivity and whether the individual must remain inactive, or only a portion of his or her body is immobilized, as where a cast is placed on a portion of the body or one or more appendages. Therefore, there is no way to definitely quantify the length of time for inactivity before the subject compositions would be recommended, except to note that they would be beneficial in any such circumstance due to their capacity to retard the process of loss of muscle strength. The following examples further illustrate the present invention, it being understood that they are in no way intended as limiting the scope thereof.

### EXAMPLE 1

The following formulation is thoroughly mixed as a dry powder. Water is added gradually to the powder with stirring to form a suspension. Once the suspension is formed, sufficient additional water is added with stirring to bring the final volume to 12 ounces. The product is pasteurized and packaged. The 12 ounces of suspension is intended as a single dose and will provide 39.3 grams of the essential composition of the invention consisting of the first six listed ingredients, optionally including arginine.

| | | Percent of Essential |
|---|---|---|
| Ingredient | Amount in Grams | Composition |
| Whey Protein Concentrate | 10.0 | 25.5 |
| Calcium Caseinate | 3.3 | 8.4 |
| Glucose | 10.0 | 25.4 |
| Sucrose | 10.0 | 25.4 |
| Leucine | 3.0 | 7.6 |
| Arginine | 3.0 | 7.6 |
| Salt | 0.45 | |
| Lecithin | 0.25 | |
| Lactarin (Carrageenan, FMC | 0.30 | |
| Corp.) | | |
| Vanilla Flavor | 0.85 | |
| Cream Plus | 0.15 | |
| Water, q.s. 12 ounces | | |

### EXAMPLE 2

The following ingredients are combined according to conventional techniques and formed into a nutrient bar. The bar, which is intended as a single dosage, contains 33 grams of the essential composition of the invention.

| | | Percent of Essential |
|---|---|---|
| Ingredient | Amount in Grams | Composition |
| Whey Protein Concentrate | 9.0 | 27.3 |
| Calcium Caseinate | 3.0 | 9.0 |
| Glucose | 9.0 | 27.3 |
| Sucrose | 9.0 | 27.3 |
| Leucine | 2.0 | 6.1 |
| Arginine | 1.0 | 3.0 |
| Glycerin | 1.8 | |
| Lecithin | 1.3 | |
| Palm Kernal Oil | 3.0 | |
| Carrageenan | 0.4 | |
| Natural Apple Crisp | 0.4 | |
| Water | 6.5 | |
| Total | 46.4 | |

### EXAMPLE 3

The following essential ingredients are blended together as a dry powder and the resultant formulator is blended with the excipient ingredients listed in Example 1, and is formulated into a 12 ounce suspension in the manner of Example 1. The essential composition of the formulation consists of the first six ingredients. Total weight of the essential composition is 58 grams

| | | Percent of Essential |
|---|---|---|
| Ingredient | Amount in Grams | Composition |
| Whey Protein Concentrate | 12.0 | 20.7 |
| Calcium Caseinate | 4.0 | 6.9 |
| Glucose | 16.0 | 27.6 |
| Fructose | 16.0 | 27.6 |
| Leucine | 5.0 | 8.6 |
| Arginine | 5.0 | 8.6 |
| Total Essential Composition | 58.0 | 100 |

### EXAMPLE 4

The following ingredients are combined according to conventional techniques and formed into a nutrient bar utilizing the excipient ingredients shown in Example 2. The bar, which is intended as a single dosage, will contain 41 grams of the essential composition of the invention.

| | | Percent of Essential |
|---|---|---|
| Ingredient | Amount in Grams | Composition |
| Whey Protein Concentrate | 9.0 | 22.0 |
| Calcium Caseinate | 3.0 | 7.3 |
| Glucose | 22.5 | 54.9 |
| Maltodextrin | 2.5 | 6.1 |
| Leucine | 4.0 | 9.7 |
| Total Essential Composition | 41.0 | 100 |

What is claimed:

1. A composition for preventing or reducing loss of muscle strength and atrophy during an extended period of inactivity comprising a carbohydrate component comprising at least two sugars selected from the group consisting of glucose, sucrose, fructose and maltodextrin, a protein component comprising a mixture of whey protein and casein, wherein the weight ratio of the carbohydrate component to the protein component is about 1.5:1 to 2:1 by weight, and an amino acid component consisting of leucine.

2. A composition is accordance with claim 1, wherein said at least two sugars are present in about equal quantities.

3. A composition in accordance with claim 2, wherein said carbohydrate component is sucrose and glucose.

4. A composition in accordance with claim 1, wherein said protein component comprises whey protein and casein in a weight ratio of about 3:1.

5. A composition in accordance with claim 4 wherein said casein is calcium caseinate.

6. A composition in accordance with claim 1, wherein said amino acid component additionally contains arginine.

7. A composition in accordance with claim 1 wherein said carbohydrate component comprises from about 35% to about 70% by weight of said composition, and said protein component comprises from about 25% to about 35% of said composition, on a dry basis.

8. A composition in accordance with claim 1, wherein leucine is present in from about 6% to about 9% by weight of said composition, on a dry basis.

9. A. composition in accordance with claim 6, wherein leucine is present in from about 6% to about 9% by weight of said composition and arginine is present in from about 3% to about 9% of said composition, on a dry basis.

10. A composition in accordance with claim 1, wherein said composition in incorporated into a preparation for consumption, said preparation additionally containing one or more excipients selected from the group consisting of suitable flavoring and sweetening agents, suitable coloring agents, emulsifiers, fillers, binders and liquid or solid vehicles suitable for the administration of foods.

11. A preparation in accordance with claim 10, wherein said composition comprises from about 15% to about 80% by weight of said preparation.

12. A preparation in accordance with claim 11, wherein said preparation is a powder suitable for mixture with a liquid or solid food prior to consumption.

13. A preparation in accordance with claim 11, wherein said preparation is a liquid.

14. A preparation in accordance with claim 11, wherein said preparation is a nutrient bar.

15. A method of preventing or reducing loss of muscle strength and atrophy during an extended period of inactivity in an individual in need thereof comprising administering to such individual an effective amount of the composition of claim 1.

16. A method in accordance with claim 15, wherein a single dosage administered to said individual comprises from about 33 to about 58 grams of said composition.

17. A method in accordance with claim 16, wherein a single dosage administered to said individual comprises from about 39 to 47 grams of said composition.

18. A method in accordance with claim 15, wherein said single dosage is administered to said individual three times a day.
